(19) United States
(12) Patent Application Publication
(10) Pub. No.: US 2026/0151203 A1
(43) Pub. Date: Jun. 4, 2026

(54) CONTROLLER ARRANGEMENTS FOR ROBOTIC SURGICAL SYSTEMS

(71) Applicant: EndoQuest Robotics, Inc., Houston, TX (US)

(72) Inventors: Jeihan Lee, Houston, TX (US); Jiwon Choi, Houston, TX (US); Yongman Park, Houston, TX (US); Dongsuk Shin, Houston, TX (US)

(73) Assignee: EndoQuest Robotics, Inc, Houston, TX (US)

(21) Appl. No.: 19/425,265

(22) Filed: Dec. 18, 2025

Related U.S. Application Data
(63) Continuation of application No. 18/134,674, filed on Apr. 14, 2023, now Pat. No. 12,514,663.
Application No. 18/134,674 is a continuation of application No. PCT/US2022/051261, filed on Nov. 29, 2022.
(60) Provisional application No. 63/284,204, filed on Nov. 30, 2021.

(51) Int. Cl. A61B 34/37; A61B 34/00; A61B 34/30

(52) U.S. Cl. CPC A61B 34/37; A61B 34/30; A61B 34/71; A61B 2034/301

(57) ABSTRACT

A controller system for a robotic surgical system can include a first instrument controller comprising a first plurality of motors configured to steer a first attachable robotically controlled medical device, a second instrument controller comprising a second plurality of motors configured to steer a second attachable robotically controlled medical device, and a camera controller comprising a plurality of motors and configured to steer an attachable robotically controlled endoscopic camera.

### CROSS-REFERENCE TO RELATED APPLICATIONS

This application is a continuation of U.S. patent application Ser. No. 18/134,674 filed Apr. 14, 2024, which is a continuation of International Patent Application No. PCT/US2022/051261 filed Nov. 29, 2022, which claims priority to and the benefit of U.S. Provisional Application No. 63/284,204, filed Nov. 30, 2021, the entire contents of which are herein incorporated by reference in their entirety.

### FIELD

This disclosure relates to robotic surgical systems, e.g., for minimally invasive surgery including, but not limited to, endoluminal and single-site surgery.

### BACKGROUND

Minimally invasive surgery such as endoluminal and single-site robotic surgery offer significant advantages versus traditional robotic surgery. For example, in endoluminal robotic surgery, no incision need be made to access difficult to access locations within a patient's natural lumen. This dramatically reduces and/or eliminates recovery time and improves procedural safety. A single-site system reduces incisions to a minimum single-site, which reduces an otherwise larger number of incisions to provide access for certain procedures.

Certain endoluminal and single-site robotic surgical systems have been proposed. Examples of such systems and related components can be found in U.S. Pat. No. 10,881,422, as well as U.S. Patent Application Nos. US20210322046, US20210322045, US20190117247, US20210275266, US20210267702, US20200107898, US20200397457, US202000397456, US20200315645, and US201962914226, all of the above being incorporated by reference herein in their entirety.

Conventional surgical robotics and systems have generally been considered satisfactory for their intended purpose. However, there is still a need in the art for improved robotic surgical systems, devices, methods, controls, and components, especially those configured for endoluminal and single-site surgery. The present disclosure provides improvements in such areas, for example.

### SUMMARY

In accordance with at least one aspect of this disclosure, a controller system for a robotic surgical system can include a first instrument controller comprising a first plurality of motors configured to steer a first attachable robotically controlled medical device, a second instrument controller comprising a second plurality of motors configured to steer a second attachable robotically controlled medical device, and a camera controller comprising a plurality of motors and configured to steer an attachable robotically controlled endoscopic camera.

The camera controller can include a hub attachment interface to connect to a steering adapter of a robotically controlled endoscopic camera. The camera controller can be positioned above the first and second instrument controller. The camera controller can be positioned symmetrically between the first and second instrument controllers.

The camera controller can include one or more motors. The camera controller can include be configured to provide three degrees-of-freedom (DOF). The three DOF can include two DOF for bending and one DOF for axial translation. The camera controller can include one or more pushing actuators (e.g., only pushing actuators).

In certain embodiments, the system can include the robotically controlled endoscopic camera. The endoscopic camera can include a steering adapter configured to receive pushing actuation of pushing actuators of the camera controller. The steering adapter can be configured to convert pushing actuation from the pushing actuators to pulling actuation to pull steering cables of the robotically controlled endoscopic camera.

In accordance with at least one aspect of this disclosure, a robotic surgical system can include a controller system. The controller system can include any suitable controller system disclosed herein, e.g., as described above.

In accordance with at least one aspect of this disclosure, a non-transitory computer readable medium can include computer executable instruction configured to cause a computer to perform a method. The method can include receiving camera control inputs from a user input device of a robotic surgical system, correlating the camera control inputs to a motor control to provide an associated instrument movement of a medical device, and actuating one or more push motors of a camera controller based with the motor control such that the one or more push motors are configured to push one or more pushing movers of a robotically controlled endoscopic camera. In certain embodiments, actuating can include actuating five push motors to provide three degrees-of-freedom (DOF).

The method can further include actuating a first instrument controller configured to steer a first attachable robotically controlled medical device. The method can further include actuating a second instrument controller configured to steer a second attachable robotically controlled medical device.

These and other features of the embodiments of the subject disclosure will become more readily apparent to those skilled in the art from the following detailed description taken in conjunction with the drawings.

### BRIEF DESCRIPTION OF THE DRAWINGS

So that those skilled in the art to which the subject disclosure appertains will readily understand how to make and use the devices and methods of the subject disclosure without undue experimentation, embodiments thereof will be described in detail herein below with reference to certain figures, wherein:

FIG. 1 is a perspective view of an embodiment of a system in accordance with this disclosure, shown having steering adapters attached to each instrument controller and the camera controller;

FIG. 2 is an elevation view of the embodiment of FIG. 1;

FIG. 3 is a perspective view of an embodiment of a hub adapter of the embodiment of FIG. 1, shown without an endoscopic camera steering adapter attached;

FIG. 4 is an elevation view of an embodiment of a robotically controlled endoscopic camera in accordance with this disclosure; FIG. 5 is a proximal perspective view of the embodiment of FIG. 4, showing the steering adapter of the robotically controlled endoscopic camera;

FIG. 6 is a perspective view of an embodiment of a frame of the embodiment of FIG. 1, shown attached to a portion of a positioning assembly to be rotated and/or translated by the positioning assembly;

FIG. 7 is a perspective view of the frame of FIG. 6, shown in isolation of the positioning assembly;

FIG. 8 is a plan view of the frame of FIG. 7;

FIG. 9 is a side elevation of the embodiment of FIG. 7; and

FIG. 10 is a front elevation of the embodiment of FIG. 7.

### DETAILED DESCRIPTION

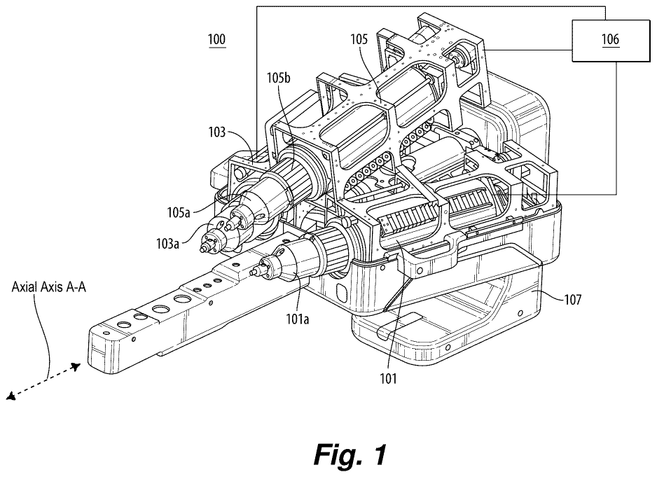

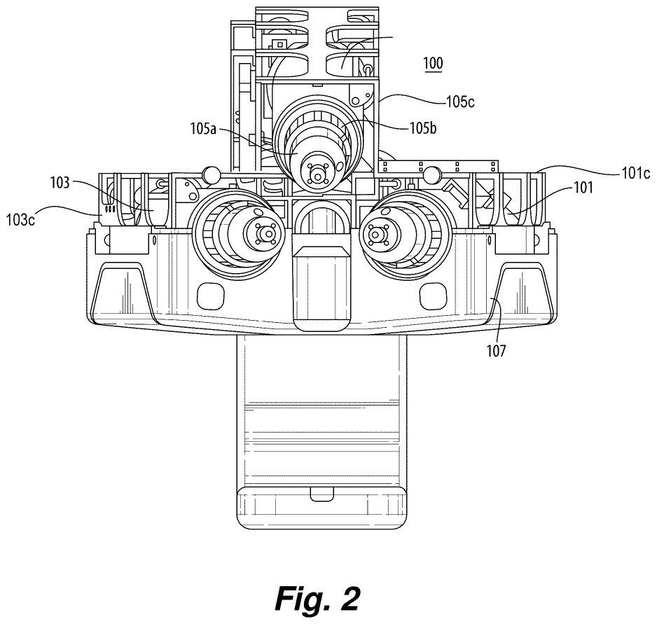

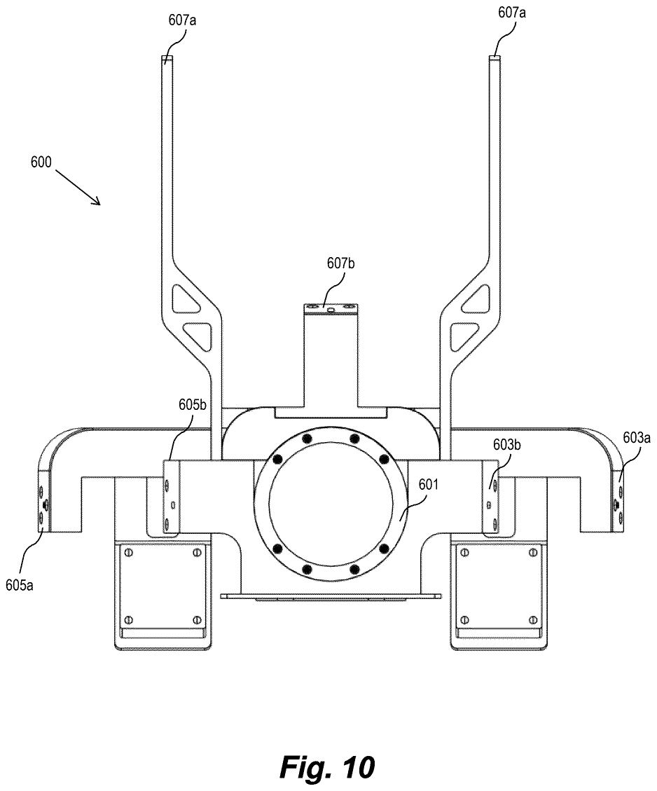

Reference will now be made to the drawings wherein like reference numerals identify similar structural features or aspects of the subject disclosure. For purposes of explanation and illustration, and not limitation, an illustrative view of an embodiment of an arrangement in accordance with the disclosure is shown in FIG. 1 and is designated generally by reference character 100. Other embodiments and/or aspects of this disclosure are shown in FIGS. 2-10.

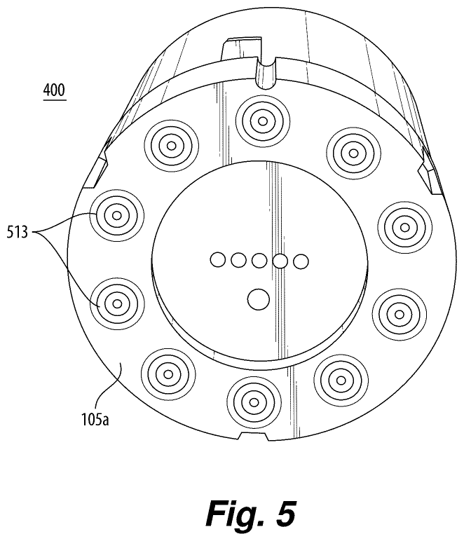

In accordance with at least one aspect of this disclosure, a controller system 100 for a robotic surgical system can include a first instrument controller 101 comprising a first plurality of motors (not shown) configured to steer a first attachable robotically controlled medical device (not shown except for steering adapter 101a), a second instrument controller 103 comprising a second plurality of motors (not shown) configured to steer a second attachable robotically controlled medical device (not shown except for steering adapter 103a). The system 100 can also include a camera controller 105 comprising a plurality of motors (not shown) and configured to steer an attachable robotically controlled endoscopic camera (e.g., as shown in FIGS. 4 and 5, steering adapter 105a shown in FIGS. 1 and 2).

Embodiments of a camera controller 105 (and/or any other suitable controllers, e.g., controllers 101, 103) can be controlled by a suitable control module 106. The control module 106 can include any suitable hardware and/or software module(s) to operate the motors of the camera controller 105 to cause steering of the camera as a function of user inputs (e.g., at a user console having one or more control inputs), for example.

The first instrument controller 101 and second instrument controller 103 can be the same or similar to the device disclosed in U.S. patent application Ser. No. 16/495,038 (e.g., see FIGS. 17-20 of U.S. patent application Ser. No. 16/495,038), previously incorporated by reference. The steering adapters disclosed herein can be the same or similar to the device disclosed in U.S. patent application Ser. No. 16/495,038 (e.g., see FIGS. 15, 16, 19, and 20 of U.S. patent application Ser. No. 16/495,038).

The controllers 101, 103, 105 can all be mounted to a positioning system 107 (e.g., mounted on a patient cart, not shown) to be moved together relative to a fixed point (e.g., a patient cart). In certain embodiments, each controller 101, 103, 105 can be angled 12 degrees from the axial axis as shown. For example, the camera controller 105 can be angled vertically 12 degrees downward, and the instrument controllers 101, 103 can be angled 12 degrees inward laterally. In such a case, an axial axis of each controller 101, 103, 105 can intersect an axial axis A-A or parallel line (e.g., as shown in FIG. 1) at an angle of 12 degrees.

The camera controller 105 can include a hub attachment interface 105b configured to connect to a steering adapter 105a of a robotically controlled endoscopic camera (e.g., camera 400 of FIGS. 4 and 5). As shown, the camera controller 105 can be positioned above the first and second instrument controllers 101, 103. The camera controller 105 can also be positioned between the first and second instrument controllers 101, 103 symmetrically. In certain embodiments, the camera controller 105 can be positioned such that the camera controller 105 and the first and second instrument controllers 101, 103 form a triangle/pyramidal shape (e.g., as shown in the view of FIG. 2).

The camera controller 105 can include one or more motors (not shown). In certain embodiments, the camera controller 105 can include five motors to provide three degrees-of-freedom (DOF). In certain embodiments, the three DOF can include two DOF for bending and one DOF for axial translation. In certain embodiments, the camera controller 105 may include four motors to provide two degrees of freedom (e.g., bending at two joints) and the positioning assembly 107 can provide an axial translation degree of freedom. However, any suitable number of motors and/or degrees of freedom are contemplated herein. For example, the camera controller 105 can include four motors configured to control two joints (e.g., to form a cobra shape).

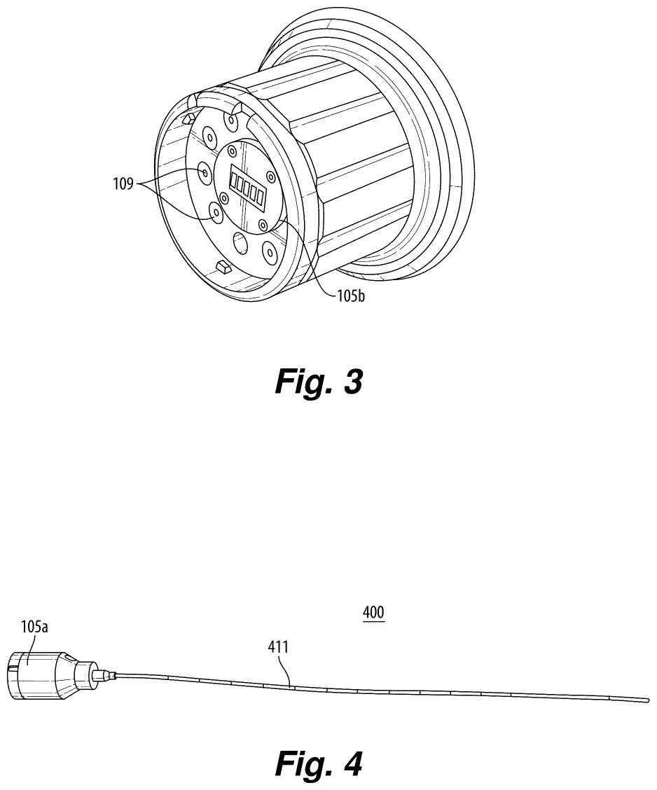

As shown in FIG. 3, the camera controller 105 can include pushing actuators 109 only. Any suitable type of actuators is contemplated herein. Pushing actuators 109 can allow swift attachment of a robotically controlled endoscopic camera 400 as no latches or other connection for the actuators 109 is required.

In certain embodiments, the system 100 can include the robotically controlled endoscopic camera 400. The endoscopic camera 400 can include a steering adapter 105a configured to receive pushing actuation of pushing actuators 109 of the camera controller 105. The steering adapter 105a can be configured to convert pushing actuation from the pushing actuators 109 to pulling actuation to pull steering cables (not shown, e.g., extending within shaft 411) of the robotically controlled endoscopic camera 400. For example, as shown in FIG. 5, the steering adapter 105a can include complementary pushing movers 513 for being pushed by the pushing actuators 109. The camera steering adapter 105a can be the same or similar to the device disclosed in U.S. patent application Ser. No. 16/495,038 (e.g., see FIGS. 15, 16, 19, and 20 of U.S. patent application Ser. No. 16/495,038).

FIG. 1 is a perspective view of an embodiment of a system 100 in accordance with this disclosure, shown having steering adapters 101a, 103a, 105a attached to each instrument controller 101, 103 and the camera controller 105. FIG. 2 is an elevation view of the embodiment of FIG. 1. FIG. 3 is a perspective view of an embodiment of a hub attachment interface 105b (e.g., a drape adapter assembly) of the embodiment of FIG. 1, shown without an endoscopic camera steering adapter 105a attached. FIG. 4 is an elevation view of an embodiment of a robotically controlled endoscopic camera 400 in accordance with this disclosure. FIG. 5 is a proximal perspective view of the embodiment of FIG. 4, showing the steering adapter 105a of the robotically controlled endoscopic camera 400.

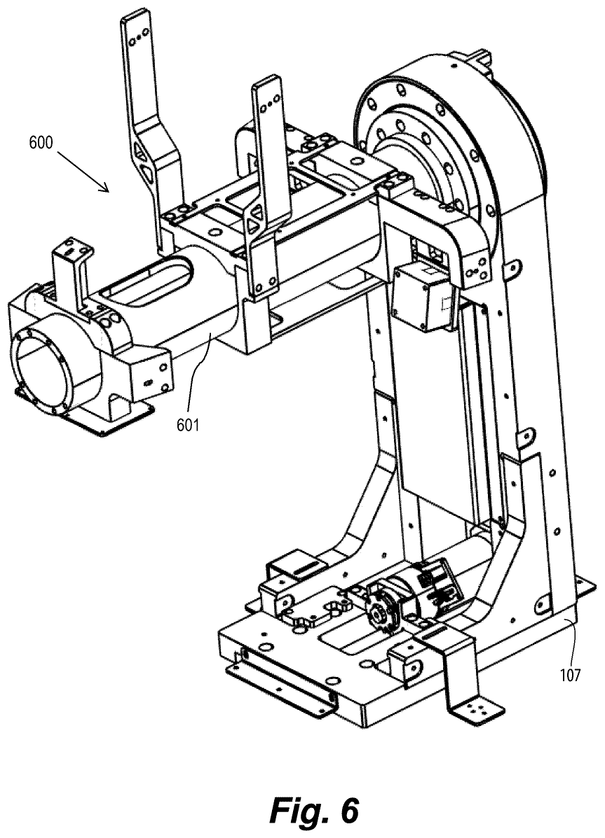

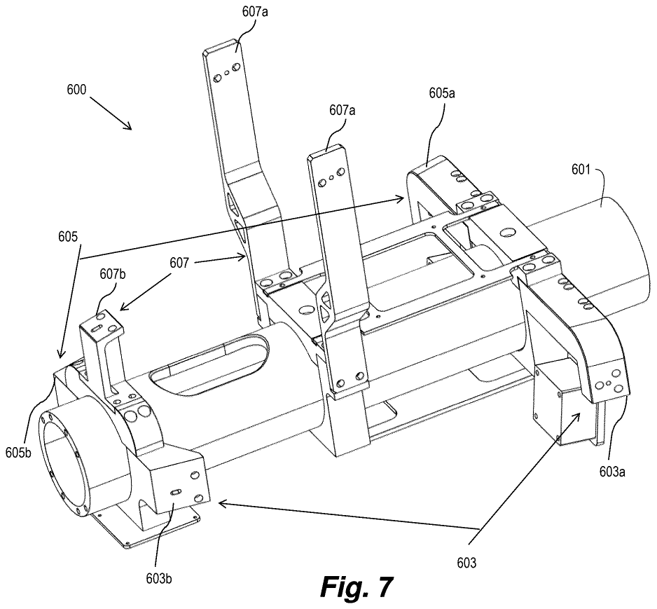

Referring to FIGS. 6-10, an embodiment of a frame 600 is shown. FIG. 6 shows the frame attached to a portion of a positioning system 107 to be rotated and/or translated by the positioning system 107. FIGS. 7-10 show the frame 600 in isolation of the positioning system (mounted to a shaft 601 which connects to the positioning system 107 to be rotated). The frame 600 can include a first lateral mount portion 603, a second lateral mount portion 605, and a vertical mount portion 607.

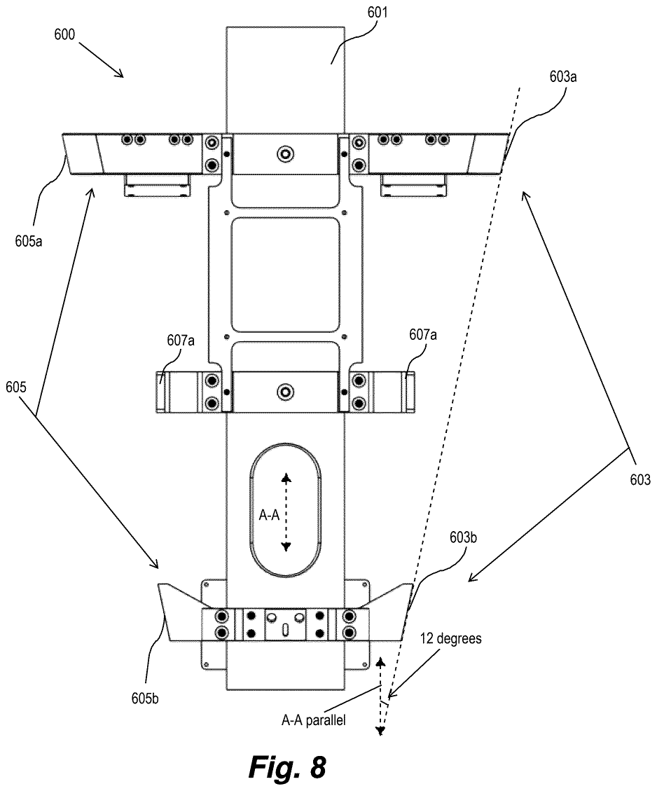

Each of the first and second lateral mount portions 603, 605 can include a proximal bracket 603a, 605a (which can be formed together or be multiple pieces), and a distal bracket 603b, 605b (which can be formed together or be multiple pieces). Each pair of proximal brackets 603a, 605a and distal brackets 603b, 605b can form two points on a plane. For example, an outer surface of each of the proximal brackets 603a, 605a and distal brackets 603b, 605b can be formed to be angled, e.g., as shown in FIG. 8. The proximal brackets 603a, 605a can extend further outward from a centerline (e.g., from the shaft 601) than the distal brackets 603b, 605b as shown, for example, to form the angled plane. In certain embodiments, each outer surface of the brackets 603a, b, 605a, b, can be angled 12 degrees relative to the axis A-A such that the planes formed are angled accordingly.

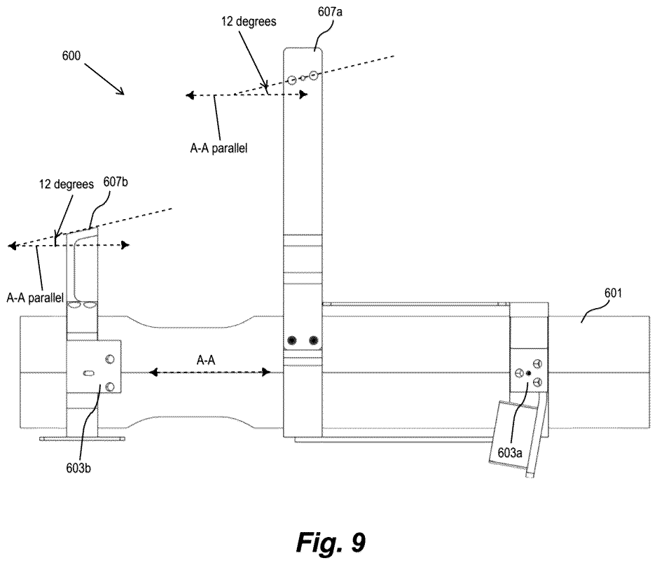

The frame 600 can include a first lateral mount portion 603, a second lateral mount portion 605, and a vertical mount portion 607. The first instrument controller 101 can be mounted to the proximal bracket 603a and the distal bracket 603b to be angled relative to the axis A-A (or parallel line), e.g., by 12 degrees or other suitable angle. The second instrument controller 103 can be mounted to the proximal bracket 605a and the distal bracket 605b to be angled relative to the axis A-A (or parallel line), e.g., by 12 degrees or other suitable angles. The vertical mount portion 607 can include a proximal brackets 607a configured and a distal bracket 607b. The camera controller 105 can be mounted to the proximal brackets 607a and the distal bracket 607b to be angled relative to the axis A-A (or parallel line), e.g., by 12 degrees or other suitable angles. The outer surface of the distal bracket 607b can be angled downward (e.g., 12 degrees as shown in FIG. 9). For example, the proximal brackets 607a can form a fork shape configured to contain the camera controller 105. The proximal brackets 607a can also include one or more fastener holes aligned at the same angle as the outer surface of the distal bracket 607b (e.g., 12 degrees as shown). In this regard, matching holes on the camera controller 105 can aid alignment of the camera controller 105 in additional to the distal bracket 607b.

As shown, e.g., in FIG. 1, each controller 101, 103, 105 can include a housing 101c, 103c, 105c configured to mount to the brackets 603a, 603b, 605a, 605b, 607a, 607b, respectively. As shown, the housings 101c, 103c, 105c can have a substantially planar shape, e.g., a square shape, to flush mount to the respective brackets 603a, 603b, 605a, 605b, 607a, 607b. The housings 101c, 103c, 105c can be fastened to the respective brackets 603a, 603b, 605a, 605b, 607a, 607b in any suitable manner (e.g., via one or more screws or bolts).

As shown, the frame 600 can be configured to have or be mounted to a shaft 601 that is part of or connected to the positioning system 107. The tube 107 can be rotated about axis A-A, for example, to provide selective rotation to all mounted controllers 101, 103, 105 simultaneously. The portion of the positioning system 107 shown in FIG. 6 can be configured to slide axially along the axis A-A to provide selective translation to all of the mounted controllers 101, 103, 105 simultaneously. Any other suitable structure and/or positioning system to accommodate the frame 600 and/or the controllers 101, 103, 105 disclosed herein is contemplated herein. Embodiments can allow a triangulation of medical instruments (e.g., having two end effectors and a camera) attached to the controllers 101, 103, 105 into a single overtube assembly (e.g., which extends substantially axially along axis A-A from the system 107) to pass the medical instruments to a distal location within a patient.

In accordance with at least one aspect of this disclosure, a robotic surgical system can include a controller system. The controller system can include any suitable controller system disclosed herein, e.g., as described above.

In accordance with at least one aspect of this disclosure, a non-transitory computer readable medium can include computer executable instruction configured to cause a computer to perform a method. The method can include receiving camera control inputs from a user input device of a robotic surgical system, correlating the camera control inputs to a motor control to provide an associated instrument movement of a medical device, and actuating one or more push motors of a camera controller based with the motor control such that the one or more push motors are configured to push one or more pushing movers of a robotically controlled endoscopic camera. In certain embodiments, actuating can include actuating one or more (e.g., five) push motors to provide three degrees-of-freedom (DOF).

The method can further include actuating a first instrument controller configured to steer a first attachable robotically controlled medical device. The method can further include actuating a second instrument controller configured to steer a second attachable robotically controlled medical device.

Embodiments can include three controllers positioned with a symmetric pattern to control robotic instruments and a videoscope. The videoscope controller can have the same configuration as an instrument controller, and the system can be designed to be retrofit onto existing patient cart systems without any or meaningful change to the patient cart/console.

Any module(s) disclosed herein can include any suitable hardware and/or software module(s) configured to perform any suitable function(s) (e.g., as disclosed herein, e.g., as described above). As will be appreciated by those skilled in the art, aspects of the present disclosure may be embodied as a system, method or computer program product. Accordingly, aspects of this disclosure may take the form of an entirely hardware embodiment, an entirely software embodiment (including firmware, resident software, micro-code, etc.), or an embodiment combining software and hardware aspects, all possibilities of which can be referred to herein as a “circuit,” “module,” or “system.” A “circuit,” “module,” or “system” can include one or more portions of one or more separate physical hardware and/or software components that can together perform the disclosed function of the “circuit,” “module,” or “system”, or a “circuit,” “module,” or “system” can be a single self-contained unit (e.g., of hardware and/or software). Furthermore, aspects of this disclosure may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of this disclosure may be written in any combination of one or more programming languages, including an object oriented programming language such as Java, Smalltalk, C++or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of this disclosure may be described above with reference to flowchart illustrations and/or block diagrams of methods, apparatus (systems) and computer program products according to embodiments of this disclosure. It will be understood that each block of any flowchart illustrations and/or block diagrams, and combinations of blocks in any flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in any flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified herein.

Those having ordinary skill in the art understand that any numerical values disclosed herein can be exact values or can be values within a range. Further, any terms of approximation (e.g., “about”, “approximately”, “around”) used in this disclosure can mean the stated value within a range. For example, in certain embodiments, the range can be within (plus or minus) 20%, or within 10%, or within 5%, or within 2%, or within any other suitable percentage or number as appreciated by those having ordinary skill in the art (e.g., for known tolerance limits or error ranges).

The articles “a”, “an”, and “the” as used herein and in the appended claims are used herein to refer to one or to more than one (i.e., to at least one) of the grammatical object of the article unless the context clearly indicates otherwise. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

Any suitable combination(s) of any disclosed embodiments and/or any suitable portion(s) thereof are contemplated herein as appreciated by those having ordinary skill in the art in view of this disclosure.

The embodiments of the present disclosure, as described above and shown in the drawings, provide for improvement in the art to which they pertain. While the subject disclosure includes reference to certain embodiments, those skilled in the art will readily appreciate that changes and/or modifications may be made thereto without departing from the spirit and scope of the subject disclosure.

1. A controller system for a robotic surgical system, comprising:
a first instrument controller comprising a first plurality of actuators configured to steer a first attachable robotically controlled medical device;
a second instrument controller comprising a second plurality of actuators configured to steer a second attachable robotically controlled medical device;
a camera controller comprising a third plurality of actuators adapted and configured to drive respective ones of a plurality of linear pushing actuators and configured to steer an attachable robotically controlled endoscopic camera.

2. The controller system of claim 1, wherein the camera controller includes a hub attachment interface adapted and configured to connect to a steering adapter of the attachable robotically controlled endoscopic camera.

3. The controller system of claim 1, wherein the camera controller is positioned above the first and second instrument controllers.

4. The controller system of claim 1, wherein the camera controller is positioned symmetrically with respect to the first and second instrument controllers.

5. The controller system of claim 1, wherein the third plurality of actuators of the camera controller provide three degrees-of-freedom (DOF) to the attachable robotically controlled endoscopic camera.

6. The controller system of claim 5, wherein the three DOF include two DOF for bending and one DOF for axial translation.

7. (canceled)

8. The controller system of claim 1, further comprising the attachable robotically controlled endoscopic camera, wherein the steering adapter is configured to receive pushing actuation of the one or more linear pushing actuators of the camera controller, wherein the steering adapter is configured to convert pushing actuation from the one or more linear pushing actuators to pulling actuation to pull steering cables of the attachable robotically controlled endoscopic camera.

9. A robotic surgical system, comprising:
a controller system, comprising:
a first instrument controller comprising a first plurality of actuators configured to steer a first attachable robotically controlled medical device;
a second instrument controller comprising a second plurality of actuators configured to steer a second attachable robotically controlled medical device; and
a camera controller comprising a third plurality of actuators adapted and configured to drive respective ones of a plurality of linear pushing actuators and configured to steer an attachable robotically controlled endoscopic camera.

10. The robotic surgical system of claim 9, wherein the camera controller includes a hub attachment interface adapted and configured to connect to a steering adapter of the attachable robotically controlled endoscopic camera.

11. The robotic surgical system of claim 9, wherein the camera controller is positioned above the first and second instrument controllers.

12. The robotic surgical system of claim 9, wherein the camera controller is positioned symmetrically with respect to the first and second instrument controllers.

13. The robotic surgical system of claim 9, wherein the third plurality of actuators of the camera controller provide three degrees-of-freedom (DOF) to the attachable robotically controlled endoscopic camera.

14. The robotic surgical system of claim 13, wherein the three DOF include two DOF for bending and one DOF for axial translation.

15. (canceled)

16. The robotic surgical system of claim 9, further comprising the attachable robotically controlled endoscopic camera, wherein the steering adapter is configured to receive pushing actuation of the plurality of linear pushing actuators of the camera controller, wherein the steering adapter is configured to convert pushing actuation from the plurality of linear pushing actuators to pulling actuation, to pull steering cables of the attachable robotically controlled endoscopic camera.

17. A non-transitory computer-readable medium, comprising computer executable instruction configured to cause a computer to perform a method, the method comprising:
receiving camera control inputs from a user input device of a robotic surgical system;
correlating the camera control inputs to a motor control to provide an associated instrument movement of a medical device; and
actuating one or more push motors of a camera controller based on the motor control so that the one or more push motors are configured to push one or more linear pushing movers of a robotically controlled endoscopic camera.

18. The non-transitory computer readable medium of claim 17, wherein actuating includes actuating one or more push motors to provide three degrees-of-freedom (DOF) to the robotically controlled endoscopic camera.

19. The non-transitory computer readable medium of claim 17, wherein the method further includes actuating a first instrument controller configured to steer a first attachable robotically controlled medical device.

20. The non-transitory computer readable medium of claim 17, wherein the method further includes actuating a second instrument controller configured to steer a second attachable robotically controlled medical device.

21. The non-transitory computer readable medium of claim 17, wherein the robotically controlled endoscopic camera includes a steering adapter configured to receive pushing actuation of the one or more linear pushing movers of the camera controller, wherein the steering adapter is configured to convert pushing actuation from the one or more linear pushing movers to pulling actuation to pull steering cables of the robotically controlled endoscopic camera.

22. The controller system of claim 1, wherein the first plurality of actuators is a first plurality of linear pushing actuators.

23. The controller system of claim 21, wherein the second plurality of actuators is a second plurality of linear pushing actuators.
